(19) United States
(12) Patent Application Publication
(10) Pub. No.: US 2009/0050152 A1
(43) Pub. Date: Feb. 26, 2009

(54) Device and Method for Automatically Regulating Supplemental Oxygen Flow-Rate

(76) Inventor: Mario Iobbi, Agoura Hills, CA (US)

Correspondence Address: BERLINER & ASSOCIATES, 555 WEST FIFTH STREET, 31ST FLOOR, LOS ANGELES, CA, 90013, US

(21) Appl. No.: 11/918,095

(22) PCT Filed: Apr. 12, 2006
(86) PCT No.: PCT/US06/13644

§ 371 Date: Oct. 9, 2007

Related U.S. Application Data
(63) Continuation of application No. 10/907,693, filed on Apr. 12, 2005, now abandoned.

(51) Int. Cl. A61M 16/20

(52) U.S. Cl. 128/204.23

(57) ABSTRACT

Device and method for limiting adverse events during supplemental oxygen therapy are disclosed. In the present invention, the oxygen flow between a patient and an oxygen source is controlled with a valve such as a proportional solenoid capable of constraining flow-rates within a continuous range. The flow-rate of oxygen is accurately controlled in a closed-loop with flow-rate measurements. Measures of a patient's vital physiological statistics are used to automatically determine optimum therapeutic oxygen flow-rate. Controller signal filtering is disclosed to improve the overall response and stability. The control algorithm varies flow-rates to minimize disturbances in the patient feedback measurements.

### FIELD OF THE INVENTION

The present invention relates to the supply of supplemental oxygen in respiratory therapy, and in particular provides a device and method to minimize adverse events during oxygen therapy.

### BACKGROUND OF THE INVENTION

For patients living with Chronic Obstructive Pulmonary Disease (COPD) treatment with supplemental oxygen to reverse hypoxemia can reduce pulmonary artery pressure, alleviate right heart failure, strengthen cardiac function, and increase exercise tolerance leading to an improved survival benefit (Krop, et al. 1973, Petty, et al. 1968). COPD is categorized by progressive obstruction to airflow from either emphysema and/or chronic bronchitis. As emphysema and chronic bronchitis frequently coexist, they are grouped together as COPD. Patients with various other pulmonary conditions can also benefit from treatment with supplemental oxygen. These patients generally suffer from their lungs' diminished ability for gas exchange performance, consequently reducing arterial blood oxygen concentration.

Respiratory therapy consisting of Long Term Oxygen Therapy (LTOT) has been shown to increase survival among patients with COPD. During the Nocturnal Oxygen Therapy Trial (NOTT) continuous oxygen therapy (mean 19 h/d) was compared versus 12 h/d, and showed a proportional reduction in mortality using continuous oxygen (Nocturnal Oxygen Therapy Trial Group. 1980). As well as a reduced mortality, other recognized benefits from LTOT include a decrease in hypoxia-induced elevations of hemoglobin, lower pulmonary artery pressure and vascular resistance, increased stroke volume index, improved exercise tolerance, and subjective improvement in quality of life (Medical Research Council. 1981, Selinger, et al. 1987). Despite the positive benefits of LTOT, even short periods of hypoxemia can have adverse effects leading to right ventricular hypertrophy from increased pulmonary artery pressure and pulmonary vascular resistance (Selinger, et al. 1987). Unfortunately with the present constant low-flow LTOT, the variable oxygen demand may not be well matched to the oxygen delivery.

Continuous oxygen is most commonly delivered via nasal cannula which can effectively deliver 100% oxygen. During inspiration this O₂ mixes with room air to increase the fraction of inspired oxygen (FIO₂). Adjusting the O₂ flow-rate will effect the FIO₂ such that each liter per minute approximately increases the FIO2 3 to 4% above room air (American Thoracic Society. 1995). It is not recommended to use flow-rates greater than 4 l/min for continuous oxygen therapy via nasal cannula. Higher flow-rates can be achieved through the use of facial masks. In actuality, the final FIO₂ will depend on a number of patient variables: anatomy, shunt fraction, and respiratory rate. For constant O₂ flow, the FIO₂ is inversely proportional to respiratory rate. Nevertheless, low-flow continuous O₂ is usually sufficient to increase arterial oxygen content to clinically acceptable levels.

The current prescription and reimbursement guidelines advocate using LTOT if a COPD patient has a resting PaO₂<55 mmHg, or PaO₂<59 mmHg if exhibiting signs of tissue hypoxia (American Thoracic Society. 1995). Hypoxemia only during exertion or sleep can be sufficient to prescribe supplemental O₂ for those settings. Using arterial blood gas (ABG), the resting PaO₂ is measured after 30 min of breathing room air. While the patient remains at rest, the oxygen flow rate is slowly titrated to achieve a SpO₂>90% as measured by oximetry. The oximetry should be calibrated against the initial ABG at rest. Further exercise testing can be performed during tasks such as a timed walk, treadmill or bicycle at a patient's normal pace. In general the guideline suggests increasing O₂ resting flow rate by 1 l/min for either exercise or sleep hypoxemia. This type of fixed regimen therapy does not account for natural fluctuations during daily activities and could promote significant periods of undocumented hypoxemia.

Recent outpatient studies utilizing ambulatory pulse oximetry have confirmed the existence of hypoxemic periods despite LTOT. Over the course of daily activities, corroborating studies revealed on average approximately 25% of the monitored period was spent with a SpO₂<90% (Morrison, et al. 1997, Sliwinski, et al. 1994, Pilling, et al. 1999). There was also poor correlations between either the guideline's resting SpO₂ or exercise SpO₂ to the time spent with SpO₂<90% (Fussell, et al. 2003). These findings highlight a critical shortcoming under the current fixed respiratory therapy. LTOT patients spend a significant undocumented percentage of time below the established saturation threshold, SpO₂>90%. Considering that even brief periods of hypoxemia can lead to right ventricular hypertrophy, this would indicate patients are not maximizing the full potential benefit from their oxygen therapy. These adverse events can not be managed with constant low-flow LTOT.

Many ‘Demand’ systems have been reported and are commercially available to increase the oxygen efficiency during supplemental oxygen therapy. For instance U.S. Pat. No. 6,220,244 discloses a device to regulate and conserve oxygen delivery to a patient. Such systems which depend upon delivering oxygen only during inspiration are termed ‘Demand’ delivery systems. Theses systems do not seek to improve the therapeutic efficacy of supplemental oxygen treatment, but minimize the gas consumption. Another ‘Demand’ method has been disclosed which regulates the dose of oxygen during inspiration in response to the measured patient oxygen saturation. In U.S. Pat. No. 6,532,958, a two state valve turns on and off a flow of oxygen, and the time duration of flow is determined by a controller. U.S. Pat. No. 6,561,187, No. 6,470,885, and, No. 6,371,114 disclose similar dose-time varying control methods. Using a two stage, on/off valve, these systems can only deliver a static flow-rate of oxygen. The shortcomings of these time dependent systems are the variations in triggering at the onset of inspiration. Studies have found significant differences in efficacy using several ‘Demand’ delivery systems (Roberts, et al. 1996, Fuhrman, et al. 2004). The disparity may also be explained by variations not only in the triggering but also the type of oxygen bolus delivered.

Other relevant prior art includes U.S. Pat. No. 6,142,149 to Steen, which describes a method for controlling the flow during supplemental oxygen therapy. The method disclosed involves automatically regulating the delivery of oxygen to a patient with discrete incremental changes in flow. This control system can lead to poor matching with patient oxygen need. The incremental controller response can create system instability or poor matching with excessive lag time. To obtain optimum system tuning, the present invention provides a continuous range of flow-rates to quickly correct any disturbance measured from the patient. This is not accomplished with the inadequate control scheme disclosed by Steen.

U.S. Pat. No. 6,675,798 also describes a control method for regulating the oxygen flow based upon the measured dissolved concentration of oxygen in the blood. In the systems disclosed, there is no provision made to include a feedback measurement to the controller regarding the absolute flow-rate to the patient. The method only provides a mechanism to offer relative changes in flow. It is important to measure and regulate the absolute flow-rate to provide safe limits. Excessive flow rates can cause irritation and lead to issues specifically when using nasal cannula. Furthermore, without feedback information in the control algorithm regarding absolute flow-rate, the system can not readily accommodate any variability in the oxygen source.

Altogether, the aforementioned prior art do not address signal conditioning the patient feedback measurement to the controller. High frequency changes can lead to potentially harmful instability in the control algorithm. More robust and effective control is possible through the use of deliberate signal conditioning. Moreover, the previously disclosed methods base the oxygen control method entirely, or at least in part, dependent on pulse oximetry. Nevertheless, other patient vital statistics can be measured to gauge the patient's respiratory function. Information regarding the patient heart rate, respiratory rate, and levels of O₂ and CO₂ can serve as important physiological measures to indicate patient distress. For instance, respiratory rate can be measured using strain gauges placed along a patient's chest. These sensors can detect when a person inhales and exhales to determine respiratory rate. In addition, measurements regarding the amount of O₂ and CO₂ can be obtained via non-invasive transcutaneous monitors or pulse oximetry. Any of these measurements can be equally important in determining adverse events during supplemental oxygen therapy. For instance, prolonged periods with supplemental oxygen therapy can depress respiration in COPD patients or lead to excessive levels of CO₂. Such adverse events are identifiable with alternative measures such as transcutaneous CO₂.

### REFERENCES

American Thoracic Society. 1995. Standards for the diagnosis and care of patients with chronic obstructive pulmonary disease. Am J Respir Crit Care Med. 152; S77-S120.
Fuhrman C, Chouaid C, Herigault R, et al. 2004. Comparison of four demand oxygen delivery systems at restand during exercise for chronic obstructive pulmonary disease. Respir Med. 98(10); 938-44.
Fussell K M, Ayo D S, Branca P, et al. 2003. Assessing need for long-term oxygen therapy: a comparison of conventional evaluation and measures of ambulatory oximetry monitoring. Respir Care. 48(2); 115-119.
Krop A. D, Block A J, and Cohen E. 1973. Neuropsychiatric effects of continuous oxygen therapy in chronic obstructive pulmonary disease. Chest 64; 1317-322.
Medical Research Council. 1981. Long-term domiciliary oxygen therapy in chronic hypoxic cor pulmonale complicating chronic bronchitis and emphysema: report of the Medical Research Council Working Party. Lancet. 1; 681-686.
Morrison D, Skwarski K M, MacNee W. 1997. The adequacy of oxygenation in patients with hypoxic chronic obstructive pulmonary disease treated with long term domiciliary oxygen; Respir Med. 91(5); 287-291.
Nocturnal Oxygen Therapy Trial Group. 1980. Continuous or nocturnal oxygen therapy in hypoxemic chronic obstructive lung disease. Ann. Intern. Med. 93; 391-398.
Petty T L, and Finigan M M. 1968. Clinical evaluation of prolonged ambulatory oxygen therapy in chronic airway obstruction. Am. J. Med. 45; 242-252.
Pilling J, and Cutaia M. 1999. Ambulatory oximetry monitoring in patients with severe COPD. Chest. 116; 314-321.
Roberts C M, Bell J, Wedzicha J A. 1996. Comparison of the efficacy of a demand oxygen delivery system with continuous low flow oxygen in subjects with stable COPD and severe oxygen desaturation on walking. Thorax. 51(8); 831-4.
Selinger S R, Kennedy T P, Buescher P, et al. 1987. Effects of removing oxygen from patients with chronic obstructive pulmonary disease. Am Rev Respir Dis 136; 85-91.
Sliwinski P, Lagosz M, et al. 1994. The adequacy of oxygenation in COPD patients undergoing long-term oxygen therapy assessed by pulse oximetry at home. Eur Respir J. 7(2); 274-278.

### SUMMARY OF THE INVENTION

The present invention provides a device and method for automatically controlling the flow-rate to a patient during supplemental oxygen therapy. The control device and method described herein is comprised of the following components:

a valve providing a continuous range of constraint to the flow-rate between and an oxygen source to a patient;

a sensor providing feedback measurement of the absolute oxygen flow-rate delivered to the patient;

a sensor providing patient feedback measurement of a vital physiological statistic;

a signal filter to condition the controller feedback response; and

a controller which determines the oxygen flow-rate based upon the patient feedback measurement.

Within the scope of the present invention, vital physiological statistic is used to refer to the feedback measurement with regards to the patient's respiratory function. The possible patient feedback measurements are understood to include but not limited to one or more of the following: heart rate, respiratory rate, blood or tissue levels of CO₂, and blood or tissue levels of O₂.

In one aspect of the present invention, the determination of flow-rate is made by the controller on the basis of a feedback measurement regarding the patient's vital physiological statistics. In the closed-loop controller provided, the flow-rate is regulated as to correct for disturbances in the patient feedback measurement. The aim is to minimize any deviations from the predetermined set value, and prevent adverse events during oxygen therapy. Specifically the oxygen flow-rate delivered to the patient is changed subject to the difference between the patient feedback measurement and a predetermined set-point value. This difference and it's variation in time can be used to calculate to the optimal oxygen flow-rate. One means of implementing the algorithm to compute the flow-rate is described herein in the detailed description of the preferred embodiments. Further, the oxygen flow-rate to the patient can be varied within a continuous range via constraint of the flow regulating valve.

Another aspect of the invention provides for a feedback measurement of flow-rate to establish a closed-loop control around the flow regulating valve. This feature ensures flow-rates are absolutely determined and limited between minimum and maximum safety limits. Further, a default flow-rate is provided if an error is detected in the patient feedback measurement. In addition, the disclosed configuration allows for use with a variety of oxygen sources. In one embodiment, the method can be implemented as a stand alone device regulating flow between any type of commercially available oxygen source to the patient. The present invention also provides for the implementation of the method as an integrated component of the oxygen delivery system.

In one particular embodiment of the present invention, transcutaneous O₂ or CO₂ measurements are used as the patient feedback measurement. This routine non-invasive measurement can be obtained from commercially available units which measure the level of O₂ or CO₂ in tissue directly across the patient's skin. Particularly, CO₂ measurements can be important in COPD patients during supplemental oxygen therapy. They are predisposed to adverse events from an excess retention of carbon dioxide. The automated controller can be implemented to respond to disturbances in the patient transcutaneous CO₂ measurement by regulating the flow-rate of oxygen. Similarly, the disclosure provides that the transcutaneous O₂ measurement can also be used as the patient feedback measurement.

In another particular embodiment of the present invention, the measurement from ambulatory oximetry is used to automate the O₂ flow rate control. A closed-loop flow-rate controller is disclosed capable of following a patient's daily fluctuations in oxygen demand, minimizing the potential for undocumented adverse hypoxemic events. The method can be implemented to develop a feedback flow control for LTOT utilizing commercially available ambulatory oximetry. From oximetry data, the O₂ flow-rate could be automatically adjusted to meet a patient's changing need. The overall aim is to create a closed-loop flow control system for patients using LTOT capable of preventing significant adverse hypoxemic events.

### BRIEF DESCRIPTION OF THE DRAWINGS

FIG. 1 is a diagram of the system schematic.

FIG. 2 is a block diagram of the controller safety logic.

FIG. 3 is a schematic of the preferred embodiment for the flow-rate control algorithm.

FIG. 4A is a plot of a representative oxyhemoglobin disassociation curve.

FIG. 4B is a plot of a representative patient saturation flow-rate step response.

### DETAILED DESCRIPTION OF THE INVENTION

The present invention provides a device and method for automatically controlling the flow-rate during supplemental oxygen therapy in order to minimize adverse events as described herein and illustrated in the accompanying drawings.

In the context of the present invention, an ‘adverse event’ is a disturbance in the patient vital physiological measurement away from the predetermined target value. The present invention will adjust the oxygen flow-rate in response to the patient feedback measurement. One embodiment of the present invention provides for using the level of O₂ at least in part to automatically control the oxygen flow-rate. Likewise, another embodiment further utilizes transcutaneous CO₂ as a patient feedback measure. As mentioned above, supplemental oxygen therapy in patients can lead to a potentially harmful accumulation of CO₂. Measures such as heart rate and respiratory rate can also in part signal patient distress. In the present invention, the flow-rate of oxygen is automatically regulated to minimize any such adverse event.

The present invention provides a closed-loop control of the oxygen flow-rate delivered to a patient. Information from a patient feedback sensor is used to automatically compute the optimal O₂ flow-rate. In addition, a signal filter is provided to condition the controller feedback response. A second closed-loop from a flow feedback sensor is used to absolutely determine the flow-rate delivered to the patient. The oxygen flow-rate is constrained via a flow regulator valve capable of a continuous range of constraint. The valve constraint is set by an output signal from the controller.

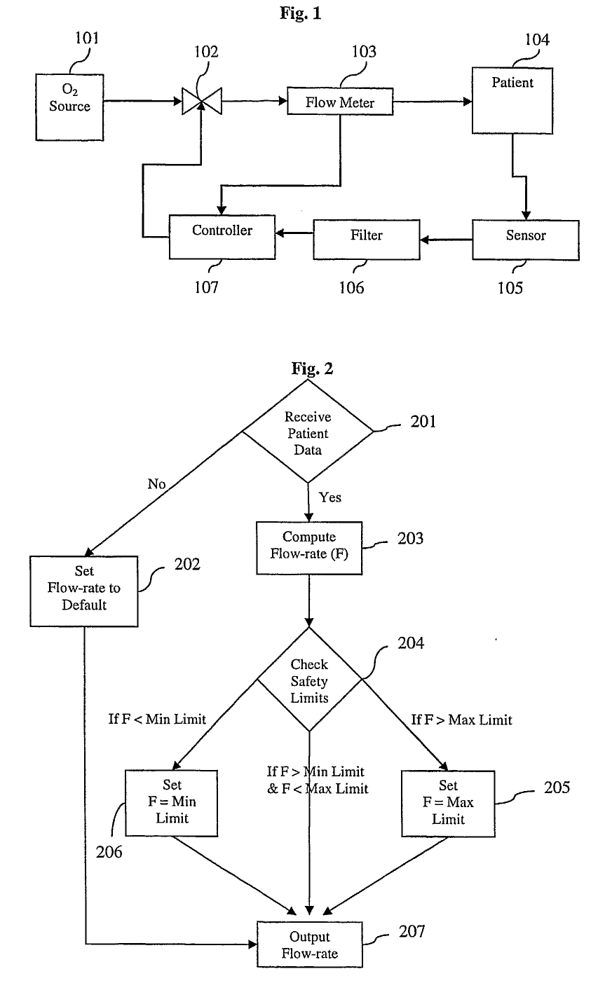

FIG. 1 depicts the general schematic of the preferred embodiment for the present invention. The oxygen source 101 is understood to include any of the various commercial systems available such as but not limited to gas cylinders, liquid oxygen, and condensers. The present invention is also not specific to the particular use whether it occur at home, hospital environment, or a portable setting. As a stand-alone device, the present invention can be incorporated between the oxygen source 101 and the patient 104 to provide automatic flow-rate control. Further, one embodiment of the present invention provides the control method be implemented directly into the system of the oxygen source 101. In the preferred embodiment, a proportional solenoid valve 102 is placed directly across the tubing connected between the oxygen source 101 and the patient 104. The proportional solenoid valve 102 can be externally regulated by either voltage or current and determine the constraint to oxygen the flow-rate. The proportional solenoid valve 102 is capable of a continuous range of constraint. Any flow regulating component may be used to replace the valve 102 provided it have the capability similar to a proportional control valve. A flow meter 103 or other similar sensor able to measure the flow-rate of oxygen delivered to the patient is placed in series with the valve 103. Feedback information regarding the measured flow-rate from the flow meter 103 is communicated to the controller 107.

Feedback measurement from the patient sensor 105 is the basis for the regulation of the oxygen flow-rate. Primary emphasis to the selection of the patient vital statistic of interest depends on the particular patient circumstance. For instance, a patient with COPD under supplemental oxygen therapy may have fluctuations in their arterial oxygen saturation. This can be measured with a pulse oximetry sensor or possibly also transcutaneous O₂ sensor. However, the present invention is not limited to patients with COPD. Other patient conditions which benefit from supplemental oxygen therapy are also provided within the scope of the present invention. Various different available sensors can be employed to measure vital physiological statistics such as heart rate, respiratory rate, tissue or blood levels of CO₂, or tissue or blood levels of O₂. Any of these can be selected to serve as the patient feedback sensor 105. In the preferred embodiment, the signal from the patient feedback sensor 105 is conditioned by the filter 106. The aim of the filter 106 is to improve the robustness of the present invention to errors in the patient feedback measurement. A person skilled in the art can implement various forms of signal filters such as low pass filter to eliminate any high frequency components from the signal. These filters are commonly implemented either as analogue or digital forms. Filtering improves the controller performance and stability over the allowable range of measured feedback response. Further, a weighted average filter can suppress the effect of sporadic artifact measurement. The conditioned signal is then communicated to the controller 107. In another embodiment of the present invention, the signal filter is used to condition the output between the controller 107 and the flow regulating valve 102. This alternate configuration places the filter 106 after the controller 107 to ensure a stable flow from the oxygen source 101 to the patient 104.

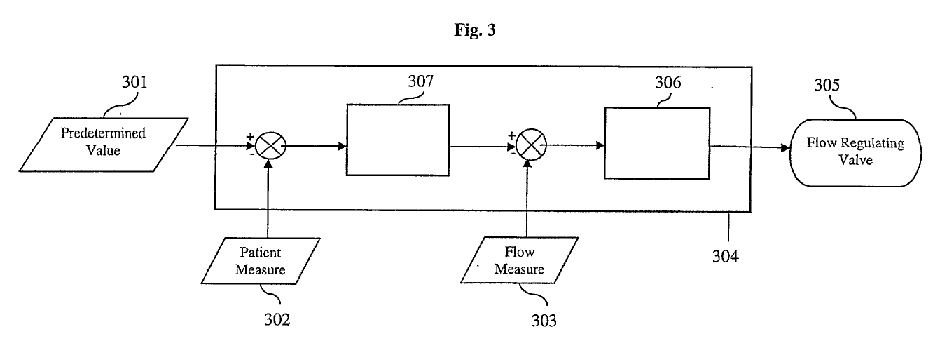

As provided by the present invention, the preferred embodiment of the controller 107 is a microprocessor to digitally compute the optimum oxygen flow-rate. The present invention can also be created as an analogue system composed of discrete circuits. Two feedback inputs are linked to the controller 107, and the output signal drives the flow regulator valve 102. In addition, the controller 107 may interact with a display unit to present and record system data. The controller 107 logic and computing algorithm are depicted in FIG. 2 and FIG. 3 respectively.

FIG. 2 is a block diagram of the controller safety logic. Several steps are taken to ensure the oxygen flow-rate to the patient always remains within allowable limits. Receipt of a valid patient feedback measurement must be verified 201 prior to computing the flow-rate 203. If no valid measurement is received, a given default flow-rate is established 202. Otherwise, the computed flow-rate is evaluated against a maximum and minimum limit 204. In the case that the maximum limit is exceeded, the flow-rate is set to the maximum limit 205. If the minimum limit is exceeded, the flow-rate is set to the minimum limit 206. Otherwise no corrective action is taken, and the flow-rate is determined 207. The default flow-rate, maximum limit, and minimum limit are all parameter given to the controller.

FIG. 3 is an illustration of the preferred embodiment for the control algorithm. The closed loop control 304 has inputs from the patient feedback measurement 302 and the flow meter measurement 303. Disturbances in the patient feedback measurement 302 are compared against a predetermined target value 301. The difference between the target value 301 and the patient feedback 302 are used to compute the optimal oxygen flow-rate 307. The optimum flow-rate is then compared against the actual measured flow-rate 303 and the difference is used to compute the output signal 306 to the flow regulating valve 305. In the preferred embodiment of the control algorithm, the closed-loop computations 306 and 307 are accomplished using a proportional, integral, and differential gain commonly known as a PID controller. This type of control system is characterized for its quick response and disturbance suppression with no steady state error. Further, the automated flow-rate controller of the present invention will vary the flow within a continuous range as to minimize any adverse events during therapy. Each computation 306 and 307 would have their distinctive PID gain parameters to optimize tuning response.

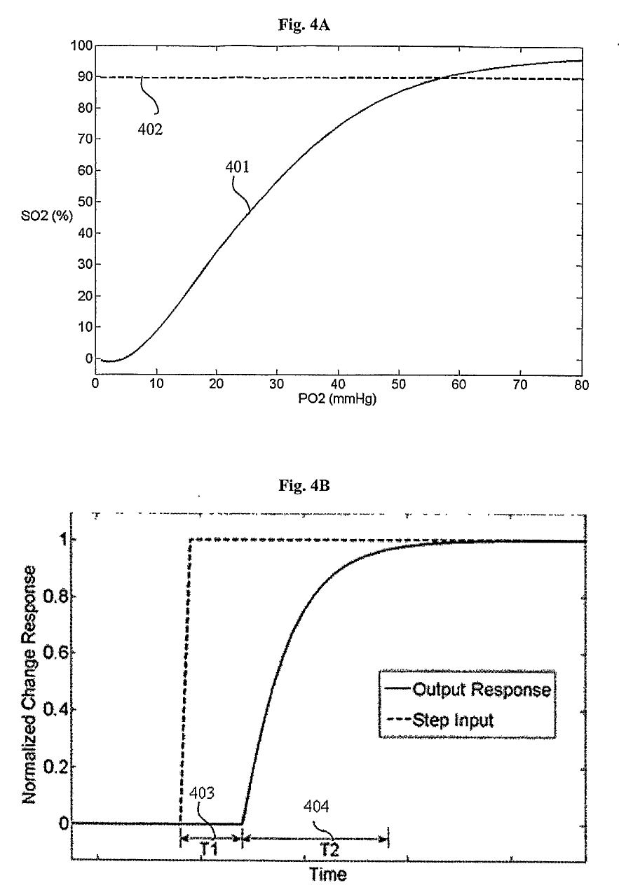

The predetermined target value 301 is a parameter given to the controller. For the preferred embodiment, the target value 301 is represented by a point on the oxyhemoglobin disassociation curve 401 represented in FIG. 4A. This value 301 is approximately 90% arterial oxygen saturation corresponding to the established threshold 402 from the medical guidelines. Below this threshold the oxygen saturation begins to change more rapidly. The PID gain parameters are critical in determining the speed and stability of the controller response to fluctuations in the patient feedback measurement. FIG. 4B depicts a representative patient response to a step increase in flow-rate. Two distinct phases are evident in the patient response. The time from the step until the patient response begins to change is known as the dead-time 403. Then the time from the onset of change until the response becomes stable is referred to as the lead-time. Ultimately these two parameters 403 and 404 will determine the optimum PID gains. Various other methods are also commonly known to establish optimal tuning for a PID closed-loop controller.

1. A device for automatically regulating the flow-rate of supplemental oxygen during respiratory support with supplemental oxygen therapy, said device comprising of:
a valve capable of a variable constraint to the flow-rate between an oxygen source and a patient, the valve is linked to a controller which determines the amount of constraint;
a sensor for measuring the oxygen flow-rate delivered to the patient as governed by said valve and configured to communicate this feedback measurement to the controller;
a sensor measuring at least one of a patient's vital physiological statistics and configured to communicate this feedback measurement to the controller;
a signal filter to condition the controller feedback response;
a controller that varies the oxygen flow-rate to the patient based upon the feedback measurement from the patient, the controller determines the change in flow-rate using the differences between said patient feedback measurement and a predetermined target set-point, the oxygen flow-rate is accurately adjusted via a closed-loop control of the valve constraint with said feedback flow-rate measurement.

2. The device according to claim 1, wherein the patient sensor measures one or more of the following vital physiological statistics: tissue or blood levels of O2, tissue or blood levels of CO2, respiratory rate, and heart rate.

3. The device according to claim 1, wherein the patient measurement is supplied via a pulse oximeter device.

4. The device according to claim 1, wherein the valve and flow-rate sensor can be combined into a single flow regulator.

5. The device according to claim 1, wherein the device is used in conjunction with any of the following oxygen sources: concentrator, gas cylinder, liquid oxygen including continuous and demand delivery systems.

6. The device according to claim 1, wherein the flow-rate determined by the controller is subject to predetermined maximum and minimum limits, including a default value when an error is detected in the patient feedback measurement.

7. The device according to claim 1, further comprising a display unit that interacts with the controller to present and record controller operation.

8. A method for automatically regulating the flow-rate of supplemental oxygen during respiratory support with supplemental oxygen therapy comprising:
adjusting the flow of oxygen between a patient and an oxygen source with a valve capable of a variable flow rate constraint, the amount of constraint determined by a controller;
measuring the oxygen flow-rate with a sensor between the patient and the oxygen source, and communicating this feedback measurement to the controller;
obtaining a measurement from a sensor regarding the patient's vital physiological statistics, and communicating this feedback measurement to the controller;
signal filtering to condition the controller feedback response;
the controller varies the oxygen flow-rate based upon the feedback measurement from the patient, the controller determines the change in flow-rate using the differences between said patient feedback and a predetermined target set-point, the oxygen flow-rate is accurately adjusted via a closed-loop control of the constraint valve with said feedback flow-rate measurement.

9. The method according to claim 8, wherein the method is integrated into the oxygen source and adapted to automatically regulate flow-rate to the patient.

10. The method according to claim 8, wherein the patient sensor measures one or more of the following vital physiological statistics: tissue or blood levels of O2, tissue or blood levels of CO2, respiratory rate, and heart rate.

11. The method according to claim 8, wherein the patient measurement is supplied via a pulse oximeter device.

12. The method according to claim 8, wherein the valve and flow-rate sensor can be combined into a single flow regulator.

13. The method according to claim 8, wherein the method is used in conjunction with any of the following oxygen sources: concentrator, gas cylinder, liquid oxygen including continuous and demand delivery systems.

14. The method according to claim 8, wherein the flow-rate determined by the controller is subject to predetermined maximum and minimum limits, including a default value when an error is detected in the patient feedback measurement.

15. The method according to claim 8, wherein the signal filtering limits the operational bandwidth to improve controller response or stability.

16. The method according to claim 8, wherein the signal filtering acts on the patient feedback measurement between the sensor and the controller.

17. The method according to claim 8, wherein the signal filtering acts on the controller output signal between the controller and the valve.

18. The device according to claim 1 wherein the variable constraint by the valve may be controlled within a continuous range of constraint thereby enabling a continuous range of oxygen flow rate to a patient.
